(19) United States
(12) Patent Application Publication
(10) Pub. No.: US 2022/0211717 A1
(43) Pub. Date: Jul. 7, 2022

(54) TOPICAL TREATMENT OF FUNGAL INFECTIONS OF THE HAIR, SKIN, AND NAILS

(71) Applicant: Garry Alan Katz, Brighton, MI (US)

(72) Inventor: Garry Alan Katz, Brighton, MI (US)

(21) Appl. No.: 17/247,992

(22) Filed: Jan. 4, 2021

(51) Int. Cl. A61K 31/5375; A61K 31/137; A61K 31/4174; A61K 31/496; A61K 31/4178; A61K 9/00; A61K 47/10

(52) U.S. Cl. CPC A61K 31/5375; A61K 31/137; A61K 31/4174; A61K 47/10; A61K 31/4178; A61K 9/0014; A61K 31/496

(57) ABSTRACT

This invention discloses methods of preparation and usage of novel pharmaceutical compositions for the topical treatment of resistant fungal infections of the hair, skin, and nails. The compositions targets the fungal cell membrane by inhibiting the biosynthesis of erogosterol, an important structural component of the membrane. The invention consists of combinations of ergosterol biosynthesis inhibitors selected from three different classis, allylamines, azoles, and morpholines.

### CROSS-REFERENCE

Not applicable.

### FIELD OF THE INVENTION

The invention pertains to the field of pharmaceutical science and the safe and effective topical treatment of resistant fungal infections of the, hair, skin and nails.

### BACKGROUND OF INVENTION

Fungal infections of the scalp (tines capitis) and nails (tines unguium/onychomycosis) are resistant to topical and systemic antimycotic therapy. The oral antimycotic drugs can have serious adverse reactions, drug interactions, contraindications and limited effectiveness.

Onychomycosis, a common infection in adults, is difficult to treat. All topical antimycotic drugs that have FDA indication for the treatment of onychomycosis have a low cure rate and the newer topical medications are also extremely expensive.

Tines capitis, also difficult to treat, usually requires initial systemic treatment with oral antifungal medication. Topical agents are not indicated for initial treatment.

Saadeh (U.S. Pat. No. 20170290810) describes pharmaceutical compositions containing three ergosterol biosynthesis inhibitors, one allylamine and two azole (imidazoles and triazoles). All the azoles (imidazoles and triazoles) inhibit the biosynthesis of ergosterol through inhibition of 14alpha-demethylase (FIG. 1).₁ The use of two azoles that block the same enzyme, 14alpha-demethylase, is redundant and unnecessary.

The present invention uses two or three different ergosterol biosynthesis inhibitors selected from three different classis, an allylamine, an azole, or a morpholine. Each class of ergosterol biosynthesis inhibitors block the biosysnthesis of ergosterol at different key steps. The allylamines blocks the enzyme squalene epoxidase, the azoles (imidazole and triazoles) blocks the enzyme 14alpha-demethylase and the morpholines blocks two enzymes, delta14-reductase and delta8, delta7 isomerase (FIG. 1).₁

There exists a need for a novel approach for treating relatively innocuous fungal infections of the hair, skin and nails which are more effective and safer than present treatments. This patent allows for an effective topical treatment of resistant fungal infections of the skin and nails and avoiding the problems seen with oral antimycotic medications.

### DETAILED DESCRIPTION

### A. Embodiments of the Invention

The invention is a pharmaceutical composition for the topical treatment of fungal infections of the hair, skin and nails that are difficult to treat. The invention uses two or three different classes of antimycotic medications selected from 3 classes to treat the infection. The three chemical classes of antifungal agents, an allylamine, an azole, or morpholines, blocks the biosynthesis of ergosterol from squalene at different steps. Erogosterol is an important structural component of the fungal cell membrane. The decrease in total ergosterol content of cell membrane and the buildup of sequalene in the fungus cell results in fungus cell death. The synergistic effect from these combinations make it possible to effectively treat resistant mycotic infection topically.

The two or three drug combinations can be ultra-micronized to increase penetration into the skin

The concentration the of the antifungal medications are between 0.1 to 20% depending the particular drug used and the dosage form.

The dosage is in the form of a cream, gel, lotion, nail lacquer, patch, shampoo, solution, suspension or tincture.

The medications can be applied topically to affected area daily to twice daily, as a combination product (mixed together) or applied individually.

The duration and frequency of application will depend on the type, severity and location of infection.

The following are examples of preparing and instructions for use of invention.

### Example 1

| | |
|---|---|
| clotrimazole | 0.5 g |
| terbinafine | 0.5 g |
| ethoxy diglycol | qs |
| Lipopen Ultra Cream Base | qs 100 g |

Method of preparation: Weigh or measure each ingredient. Titrate the powders together. Add sufficient quantity of ethoxy diglycol to form a smooth paste. Geometrically, incorporate the Lipopen Ultra Cream Base to final weight.

Method of application: Apply to toenail and skin around nail twice daily.

### Example 2

| | |
|---|---|
| itraconazole | 0.5 g |
| butenafine | 0.5 g |
| amoroline | 0.5 g |
| ethoxy diglycol | qs |
| Lipopen Ultra Cream Base | qs 100 g |

Method of preparation: Weigh or measure each ingredient. Triturate the powders together. Add sufficient quantity of ethoxy diglycol to form a paste. Geometrically, incorporate the Lipopen Ultra Cream Base to final weight.

Method of application: Apply morning and evening to affected area on scalp.

### Example 3

| | |
|---|---|
| ketoconazole | 2 g |
| butenafine | 0.5 g |
| ethoxy diglycol | qs |
| Dr. Bronners Tea Tree Liquid Soap | |

Method of preparation: Weigh or measure each ingredient. Triturate the powders together. Add sufficient quantity of ethoxy diglycol to form a paste. Geometrically, incorporate the Dr. Bronners Tea Tree Liquid Soap.

Method of application: Shampoo scalp, leave on for 10 to 15 minutes then wash off. Use twice weekly as needed.

### Biosynthesis of Ergosterol

### Squalene

|Squalene epoxidase

| Allyamines—Squalene epoxidase Inhibitors

|

### Squalene Epoxide

|

### Lanosterol

|14alpha-demethylase

|Azoles-14alpha-demthylase inhibitors

|(imidazole and triazole)

4,4-dimethyldimethylcholestra-8,14,24 trienol

|delta14-reductase

4,4-dimethylzymosterol Morpholines-delta14-reductase and

|delta8, delta7 isomerase

|delta8, delta7 isomerase inhibitors

|

### Fecosterol

|

### Episterol

|

### Ergosterol

### REFERENCES

1. Lemke, T., Williams, A. Roche, V., Zito, S. Foye's Principles of Medicinal Chemistry sixth edition.

1. A pharmaceutical formulation for the topical treatment of resistant dermatophytic infections of the hair, skin, and nails consisting of two or three ergosterol biosynthesis inhibitor medications selected from three different classes, an allyamine, an azole, or a morpholine.

2. The pharmaceutical composition of claim 1, wherein the composition of the allylamine drug is selected from the group consisting of, butenafine, naftifine, terbenifine and pharmaceutically suitable salts or hydrates thereof.

3. The pharmaceutical composition of claim 1, wherein the composition of the azole drug is selected from the group consisting of butoconazole clotrimazole, econazole, itraconazole, ketoconazole, luliconazole, miconazole, oxiconazole, sertaconazole, sulconazole, terconazole, tioconazole and pharmaceutically suitable salts or hydrates thereof.

4. The pharmaceutical composition of claim 1, wherein the composition of the morpholine drug is amorolfine and pharmaceutically suitable salts or hydrates.

5. The pharmaceutical composition of claim 1, wherein the dosage form is a cream, gel, patch, ointment, nail lacquer, shampoo, solution, suspension or tincture.

6. The pharmaceutical composition of claim 1, wherein the antifungal can be ultra-micronized to increase the penetration of the drug into the affected area.

7. The pharmaceutical composition of claim 1, wherein the concentrations of the antifungal medications are between 0.1 to 20%.

8. The pharmaceutical composition of claim 1, wherein the antifungal is applied topically as a combination product or applied individually, terbinafine in the morning and clotrimazole in the evening.
